(19) United States
(12) Patent Application Publication
(10) Pub. No.: US 2006/0167526 A1
(43) Pub. Date: Jul. 27, 2006

(54) Medical apparatus and system for preventing and treating diseases

(76) Inventors: Xushan Wan, San Pedro, CA (US); Long Wan, Shenyang (CN); Xiaozhe Liu, (US)

Correspondence Address: Xushan Wan, 647 W. 9th St, San Pedro, CA, 90731, US

(21) Appl. No.: 11/041,643

(22) Filed: Jan. 24, 2005

(51) Int. Cl. A61N 1/34; A61N 1/00; A61K 36/00

(52) U.S. Cl. 607/46; 424/725; 600/15

(57) ABSTRACT

Medical apparatus and system are provided for preventing and treating diseases by the medical apparatus comprising body-thing, electronic pulse device, medical magnet slices, herbal medicine gauze pads and nano-micro-biochemical-textile. The medical apparatus is made in various types such as cap, eyeshade, waistband, knee-band, wristband based on different position of human body. The medical apparatus and system are effective and safe for preventing and treating diseases such as headache, migraine, eyestrain, nervosas, insomnia, forgetful, overweight, hypertension, diabetes, cerebral thrombosis, cervical spondylosis, lumbago and seraglio, rheumatoic arthritis, scapulohumeral periarthritis.

### BACKGOUND OF THE INVENTION

1. Field of the Invention

The present invention relates to medical apparatus and system for preventing and treating diseases. More specifically, it relates to the medical apparatus comprising body-thing, electronic pulse device, medical magnet slices, herbal medicine gauze pads and nano-micro-biochemical-textile.

2. Prior Art

The method of medication has been become main one for treating diseases in modern society. But that can't satisfy needs of people to prevent and treat diseases. People still look for methods more safe, more effective, more and more ones to struggle with diseases and illnesses increased day by day. Some of non-medication methods have been used to prevent and treat diseases, such as physiotherapy, aromatic therapy, musical therapy, external therapy, magnetic therapy, acupuncture therapy, massage and tuina therapy. Many of medical and health products are accepted and liked by people. Modern high-new technology and science enter medical field more and more rapidly, such as gene technology, biological engineering, nano-meter technique.

Bergersen, Turid U.S. Pat. No. 20040098036 entitled “Device for acupuncture treatment” discloses a device for acupuncture treatment, more specifically for stimulating the acupoints BL1 and BL2 on both sides of the root of the nose and over the eyes near the roof of the nose, while one set of pads faces in towards the plane of symmetry of the bow, the other set of pads faces sideways out from the plane of the bow.

Otte, Per U.S. Pat. No. 20020007835 entitled “Method of treating opthalmalogical conditions using micro-acupuncture” discloses a method of treating ophthalmologic conditions including macular degeneration, myopia, hyperopia, diabetic retinopathy and glaucoma in a patient, using micro acupuncture comprises the invention. The method includes the steps of placing needles in the patient at key trigger points for a prescribed period of time and then removing the needles.

George H. Kramer U.S. Pat. No. 20030074020 entitled “Acupuncture treatment device and methods of use” discloses an acupuncture device for use on a selected skin surface portion of a human body. The device comprises a base sheet; one or more protrusions; a fastener; and an orienting element suitable to position the device on the selected skin surface. The invention also provides therapeutic methods comprising applying a device of the invention to a human in need of therapy.

Josef Constantin Szeles U.S. Pat. No. 20040044390 entitled “Electrode system for electric point stimulation therapy and a manipulation tool therefore” discloses an electrode system for an electric punctual stimulation therapy, which system comprises at least one stimulation electrode with a disk-like base and has an annular latter is pierced by the electric needle.

Catherine Lin-Hendel U.S. Pat. No. 20040230256 entitled “Electronic electrical and electromagnetic health enhancement and stimulation device” discloses an electronic acupressure aide and stimulating device implemented using a hand-held or palm-held electronic computing device or another computing device which may be a designated unit. That allows a practitioner to apply a pulse sequence to a set of predetermined acu-points such as those related to acupressure, acupuncture, trigger points or Jin-Shin Jyutsu, to name a few. A displayed chart related to the acu-points identifies the health condition and the pulse sequence.

Zou, Shazhou et al U.S. Pat. No. 20040102819 entitled “Portable therapeutic device and method for pain relief” discloses a method and device of reducing pain and other symptoms in a patient utilizing a non-invasive electrical stimulation device applied over acupuncture points and adjacent nerves of a patient. The device includes electronic circuitry for imparting electrical impulses to the desired acupuncture points and areas. Batteries within the device power the electric circuitry, and an operable switch on the device controls on/off and pulse frequency and amplitude.

Fred P. Swing U.S. Pat. No. 20010031989 entitled “Treatment of peripheral vascular disease, leg cramps and injuries using needles and electrical stimulation” discloses a method for healing an injury of a patient using an electrical stimulator and acupuncture needles.

Stephen A. Steponovich U.S. Pat. No. 20020010410 entitled “Patella tracking knee brace and magnets system/method” discloses a neoprene strap having loop type connector material along the length of one side to mate adjustably with a strip of hook type connector material on the other side at one end of the strap to tightly stretch the strap around the patella in a double loop. Magnets with hook type connector material attach at any desired points along the strap on the loop type connector material in contact with the skin at the desired points of application of magnetic therapy around the knee.

Johnny M. J. Lu U.S. Pat. No. 20040111005 entitled “Magnetic accessory” discloses a magnetic accessory, including a number of magnetic stones and sets of beads alternating each other in a line. The magnetic stones and the sets of beads are secured to each other to form a chain. The magnetic accessory can be worn by surrounding the wearer's body, such as neck, waist, ankle and wrist and secured to the wearer's body by the magnetic stones magnetically attracting each other. Magnets have healing purposes to improve blood circulation, strengthen revitalization of the nerves, eliminate stiffness and fatigue.

Robert Be Xie U.S. Pat. No. 20040167373 entitled “Method of acupuncture and magnetic treatment for weight loss” discloses a method for effective means of weight loss without negative or harmful side effects treatment as well as for the treatment of ailments in human patients. Treatment is achieved using a combination of acupuncture and magnets. The method comprises the steps of placing several acupuncture needles into specified locations on the human body, removing the acupuncture needles, and placing several magnets onto the same locations that the needles previously occupied. Another important object of the present invention is to provide and effective means of coping with and managing diabetes.

David Schmidt U.S. Pat. No. 20040057983 entitled “Biomolecular wearable apparatus” discloses a wearable apparatus that may include bimolecular components having orthomolecular and/or thermo magnetic levororotary action and/or thermo magnetic dextrorotatory action.

Li Cheng Yu U.S. Pat. No. 20040185121 entitled “Chinese herbal medicine composite system for use in treatment of paralysis” discloses a Chinese herbal medicine composite system for use in treatment of paralysis. Within the system different formulas are prescribed according to the body condition of the patient; the system comprising three separate Chinese herbal medical formulas.

Liqin Sun U.S. Pat. No. 20040043062 entitled “Acupoint patch” discloses a method, apparatus and method of making acupoint patches. The acupoint patch uses natural herbs as raw materials. It is made by combining traditional Chinese acupuncture therapy and contemporary transdermal controlled release technologies. It is made with natural herb extracts using contemporary transdermal absorption technologies based on the characteristics of different acupoints. They can stay on the skin for 6-8 hours, during which period natural herb essence enters blood circulation and performs a sustainable regulating function. This is a feature unrivaled by traditional acupuncture.

Morton I Hyson U.S. Pat. No. 20030139698 entitled “Medicated wrap” discloses a device for treating pain in an injured body member includes a wrap having an interior section which can be loaded with medicament.

I-Cherng Chen et al U.S. Pat. No. 20040137214 entitled “Material with surface nanometer functional structure and method of manufacturing the same” discloses a material with a surface nanometer functional structure and the method of manufacturing the same. Using the propertie's of supercritical fluids, a nanometer structure is formed on the surface of a substrate, resulting in a material with a surface nanometer functional structure. Utilizing the VLS nanowire growth method, one-dimensional and two-dimensional compound nanometer functional wire structure can be produced.

Xin Wang et al CN. Pat. No. 01102720.7 entitled “Nanometer treating device” discloses a nanometer treating device with radiation of collecting heat, magnetic pulse and human body bioelectricity control. The key point of the invention is putting nanometer combined powder on the nanometer launching board. It make the surface of the device to be full of active nanometer energy fields.

Guozhu Wang CN. Pat. No. 03236433.4 entitled “Eye-acupuncture treating device” discloses an eye-acupuncture treating device with permanent magnet and low frequency pulse. It put electrode contact on acu-point of eye around and proceed low frequency electricity treating.

There are many medical and health products with magnet and Chinese herbs in China patent. A lot of them are wear health products. Some of them are with magnet; some of them are with Chinese herbs; some of them are with magnet and Chinese herbs. For example, Qiming Yang CN. Pat. No. 00119031.8 entitle “therapy hat of Chinese herbs and magnet”; Shuangquan Xia CN. Pat. No. 94100223.3 “Comfortable magnet therapy cap”; Jianzhe Huang CN. Pat. No. 02241973.X “Eyeshade of sheltering light and benefiting sleep”; Yonghua Zhang CN. Pat. No. 97122334.3 “pressing and magnet therapy eyeshade”; Deshan Liu CN. Pat. No. 96220708.X “Meridians and collaterals and acupoint treating health hat with Chinese herbs”; Ziren Wu CN. Pat. No. 94244126.5 “Multi-function cultivating life and health cap with Chinese herbs”; Zhongrong Miu CN. Pat. No. 93202062.3 “A cap for Tranquilizing spirit and invigorating brain” (with Chinese herbs); Guozheng Wang CN. Pat. No. 92109943.6 “Medical health pillow and hat” (with Chinese herbs); Tiezhu Zhang CN. Pat. No. 99241919.0 “A wear appliance for preventing and treating computer eye-disease” (with Chinese herbs); Hengyu Yang CN. Pat. No. 93216156.1 “A abdomen band with Chinese herbs for weight loss”; Hanzhang Zhu CN. Pat. No. 90214661.0 “Waist band for health and weight loss” (with Chinese herbs bag); Yuelun Sun CN. Pat. No. 92109920.7 “Shoulder bag, waist bag, knee bag for having medical health function” (with Chinese herbs); Youcun Zhu CN. Pat. No. 94105653.8 “Meridians and collaterals cap” (with magnet and Chinese herbs); Yong Cao CN. Pat. No. 93205774.8 “Band for nourishing Qi and invigorating spirit to proceed along meridians” (with magnet and Chinese herbs).

The above prior arts have good action to prevent and treat diseases. However, the present invention is different from before. It has more advantage and more improvement than above.

Present invention is a perfect combination of the electronic pulse therapy, magnet therapy, herbal medicine therapy on acupoint and nano-micro-biochemical-textile therapy for preventing and treating diseases. It is effective, safe, and advanced technique.

### SUMMARY OF THE INVENTION

The principal object of the present invention is to provide the medical apparatus and system for preventing and treating diseases by the medical apparatus comprising body-thing, electronic pulse device, medical magnet slices, herbal medicine gauze pads and nano-micro-biochemical-textile.

It also is an object of the present invention to provide a basic structure of the medical apparatus. The basic structure of the medical apparatus comprise (1) body-thing having different size and shape adapted to be worn on the human head or eyes or waist or wrists or arms or legs or knees or shoulder; (2) at least one external electronic pulse device that can be linked with said the body-thing; (3) and the gauze pads soaked with concentrated liquid of herbal medicine that the gauze pads can be put on the surface of the magnet slices and electronic pulse contacts in the body-thing.

Another object of the present invention is to provide the different types of the medical apparatus. Which are decided by the different shape of the body-thing and the different position to be worn on the human body such as head, eyes, waist, wrist, arms, legs, knees, shoulders, ect.

Another object of the present invention is to provide the main structure of the body-thing. The body-thing is that can be divided three layer: (1) inside layer: the nano-micro-biochemical textile and its accessory—the magnet slices and the electronic pulse contacts put on the therapy acupoints; (2) middle layer: the elastic that make the magnet slices and the electronic pulse contacts to fix easy and touch the human body next to the skin closer together; (3) outside layer: the sponge and the adornment cloth.

A further object is to provide the nano-micro-biochemical-textile used in the body-thing. Which is a kind of textile having special biochemical function.

A further object is provide the technique standard of the magnet slice. Which is a kind of permanent magnet slice having medical and health function. Its diameter is 8-24 millimeter and thickness is 3-8 millimeter. Its magnetic induction intensity is 800-1800 gauss.

A further object is to provide the sandwich method of assembling permanent magnet slice. The magnet slice is fixed on the surface of the nano-micro-biochemical-textile by magnetism of another iron slice fixed on the elastic.

A further object is to provide the technique standard of the electronic pulse contact. The electronic pulse contact is a kind of electrode contact made in soft rubber material and shape of screw with head of partial sphere and screw bolt having electric conductivity. Its head diameter is 8-24 millimeter and thickness is 3-8 millimeter. The method of fixing it is by its screw bolt and screw nut with thread. The electronic pulse contact is fixed on the surface of the nano-micro-biochemical-textile by the screw bolt and screw nut and elastic.

Another object of the present invention is to provide a improved electronic pulse device. Which is divided to three parts: the main body, the link wire and another set of accessory of electronic pulse contacts.

A further object is to provide a main body structure of the electronic pulse device. The main body comprise the outer covering with the knob of mode and the knob of timer; the two control and the two control window; the frequency light, the low battery indicator, and the protection light indicator; the display screen of the electronic clock and three button of the electronic clock; the two sockets; and the interior with one battery and the collector plate of the electronic pulse device and electronic clock. The collector plate is exist technique already.

A further object is to provide the link wire of the electronic pulse device. Which is the link wire with double plugs.

A further object is to provide the another set of electronic pulse contacts. Which is 6-8 electronic pulse contacts that can be put on the other acupoints of the human body.

The foregoing object of the present invention is to provide method of making the gauze pads. The gauze is cut to small round or square slice pads and soaked with concentrated liquid of herbal medicine. The liquid is made by extracting and concentrating herbal medicine. The herbal medicine is the compound with different herbs based on different formulae. The formulated different formulae are based on the need of the different diseases.

A further object is to provide the method of herbal medicine therapy. Which is putting gauze pads soaked with concentrated liquid of herbal medicine on the surface of the magnet slices and the electronic pulse contacts when the treatment is needed.

Another object of the present invention is to provide some of therapy acupoints. The other therapy acupoints are based on the different diseases and different position of the human body.

A further object is to provide the therapy acupoints of the magnet slice and electronic pulse contacts in the medical cap. Which are ShangXing, QianDing, BaiHui, SiShenCong, HouDing, QiangJian, NaoHu, FengFu, TouLinQi, MuChuang, NaoKong, FengChi.

A further object is to provide the therapy acupoints of the magnet slice and electronic pulse contacts in the medical eyeshade. Which are TaiYang, TongZiLiao, YuYao, RanZhu, YinTang, JingMing, ChengQi, Vision field, and FengChi.

A further object is to provide the therapy acupoints of the magnet slice and electronic pulse contacts in the medical waistband. Which are HuaRouMen, ShuiFen, WaiLing, YinJiao, ShenQue, TianShu, SanJiaoShu, ShenShu, QiHaiShu, DaChangShu, YaoYangGuan, MingMen, XuanShu, XiaJiShu.

The invention is based on the theory of traditional Chinese medicine and new technology of modern science.

The theory of traditional Chinese medicine and principle of meridians have been recognized by most people in the world. Acupuncture therapy and electronic acupuncture method have been used to thousands and thousands patients in China, America, and other county. Its curative effect has been proved and confirmed by fact.

Magnet therapy has been used for more than two thousands years. Many patents of U.S. and China are about magnet therapy. Magnet therapy has been recognizing by more and more people and official.

Nanometer technique is a high-new science and technology rising in the world. Nanometer is one basic length unit in physical field. 1 nanometer is equal to one billionth meter. Scientific test prove that one material could happen surprising change in physical and chemistry characters when one common material convert into Nano-material after treated with scientific methods. The Nano-material show much more active character and has great potential energy inside which is called “quantity accumulation lead to quality change”. For instance, conductive Cu metal won't be one kind of electrical conductive material when its constructive unit reach to Nano size.

Nano-micro-biochemistry textile to be used in the invention, taken from natural mineral contain many mic-material, through high technology disposal is a production together with thermodynamics, optics, macromolecule-physics, iatrology, blood stream knowledge, textile-engineering, have the function of absorbing and transmitting the human's energy of the nature. And change it as human's biology power, then activate the cell, promote metastasis, fully adjust human's mic-circulation, elevate human's immunity, recover human's health.

Nano-micro-biochemistry textile can ameliorate human body's micro-circulation. And micro-circulation is the source of all diseases. So many expert call the Micro-circulation the second heart of the human body.

Micro-circulation is that the blood flows through the little arteriole, through the capillary, flow into the circulation of the little arteriole. Main function of it to histiocyte transport oxygen and nourishment, and take carbon dioxide and other metabolite that produced by histiocyte. When decline in Micro-circulation, the wearing out of the human body begins, too.

The micro-circulation obstacle is a main cause to producing diseases. Changes on the form of capillary, and functional change caused in change on the blood flow form, so many expert call it Micro-circulation obstacle.

The micro-circulation obstacle can cause the following results: the blood volume is relative or definitely insufficient; the metabolism is disorderly, makes the histiocyte ischemia, acidosis and produce a series of serious consequences; the internal organs implicated; the serious one may occur diffusibility intravascular coagulation.

Ordinary diseases include hypertension, coronary heart disease, cerebral thrombosis diabetes, cervical spondylosis, lumbago, rheumatism, rheumatoid arthritis, scapulohumeral periarteritis, neurasthenia, migraine.

Nano-micro-biochemistry textile products have been put on the market in Hong Kong and China.

The present invention collects advantage of above technology and products. Ingenious design, rational structure, unique advantage and advanced technique are gather together. A series of new medical apparatuses with Nano-micro-biochemical-textile, Acupoint therapy, Magnet therapy, Electronic pulse therapy, Herbal medicine therapy will be created. These new medical apparatuses will be different from any above products in main structure and design. Some of them is distinguish in comparison of patents previously.

The medical apparatuses of present providing invention can make use of bio-energy, activate cells, promote metastasis, fully adjust human's micro-circulation, elevate human's immunity, regulate Qi and blood, balance Yin and Yang, prevent and treat diseases. Each of a series of the medical apparatus is to be aimed at different diseases, different location, different acupoint, adequately use integrated function of electronic pulse acupuncture, magnetic therapy, herbal medicine therapy and nano-micro-biochemical textile to prevent and treat diseases.

A series of the medical apparatuses may be divided into many different shapes, types, sizes. The main medical apparatuses have: cap, shade, waistband, knee band, wrist band, shoulder band, back band and pillow etc.

### BRIEF DESCRIPTION OF THE DRAWINGS

FIG. 1 is a illustration of key structure of body-thing about the medical apparatuses.

FIG. 2 is a illustration of detailed structure about fixing the magnet slices and electronic pulse contacts.

FIG. 3 is a illustration of the electronic pulse device about the medical apparatuses.

FIG. 4 is a illustration of the package bottle and gauze pads soaked with concentrated liquids of herbal medicine.

FIG. 5 is a illustration of key structure and therapy acupoints in front top of the medical cap.

FIG. 6 is a illustration of key structure and therapy acupoints in back of the medical cap.

FIG. 7 is a illustration of key structure and therapy acupoints in flank of the medical cap.

FIG. 8 is a illustration of key structure and therapy acupoints in front of the medical eyeshade.

FIG. 9 is a illustration of key structure and therapy acupoints in back of the medical eyeshade.

FIG. 10 is a illustration of key structure and therapy acupoints in front of the medical waistband for losing weight.

FIG. 11 is a illustration of key structure and therapy acupoints in back of medical waistband for losing weight.

### DETAILED DESCRIPTION OF THE INVENTION

The present invention pertains to apparatus and system for preventing and treating diseases. The apparatuses comprise the body-thing, electronic pulse device, medical magnet slice, gauze pads soaked with concentrated liquid of herbal medicine and nano-micro-biochemical textile. The body-thing are made in various types such as cap, eyeshade, waistband, etc. based on the different position of human body. The system and apparatus are effective and safe for preventing and treating diseases such as headache, migraine, eyestrain, hypertension, coronary heart disease, cerebral thrombosis, diabetes, cervical spondylosis, lumbago, rheumatism, rheumatoid arthritis, scapulohumeral periarteritis, neurasthenia, migraine, insomnia, forgetful, overweight, etc.

Above medical apparatus's main structure is divided three parts. One part is called body-thing due to touching with human body next to the skin such as cap, eyeshade, waistband, wristband, knee-band, shoulder-pad, etc. One part is a electronic pulse device. Another part is package bottle within gauze pads soaked with concentrated liquid of herbal medicine.

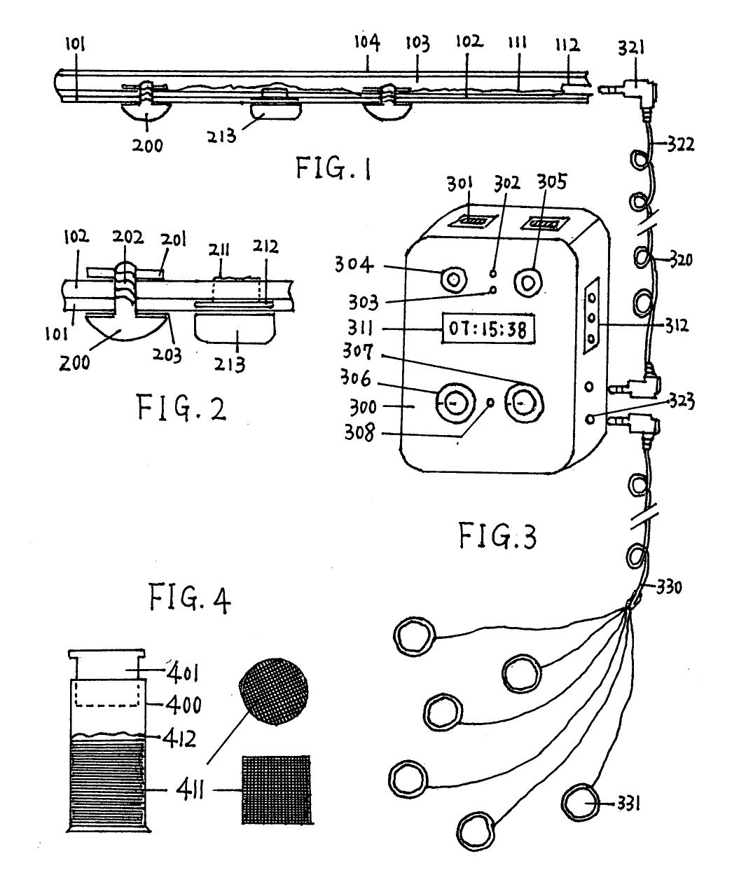

FIG. 1 is a illustration of key structure of body-thing about the medical apparatuses. The body-thing can be divided three layer: (1) inside layer: the nano-micro-biochemical textile 101 (FIG. 1) and it's accessory such as the magnet slices 213 (FIG. 2) and the electronic pulse contacts 200 (FIG. 2); they are main parts for preventing and treating function. (2) middle layer: the elastic 102 (FIG. 1). It is a framework that make the magnet slices 213 and the electronic pulse contacts 200 to fix easy and touch the human body next to the skin closer together. (3) outside layer: the sponge 103(FIG. 1) and the adornment cloth 104(FIG. 1). The sponge has function of protection and softness. The adornment cloth has effect of adorning appearance.

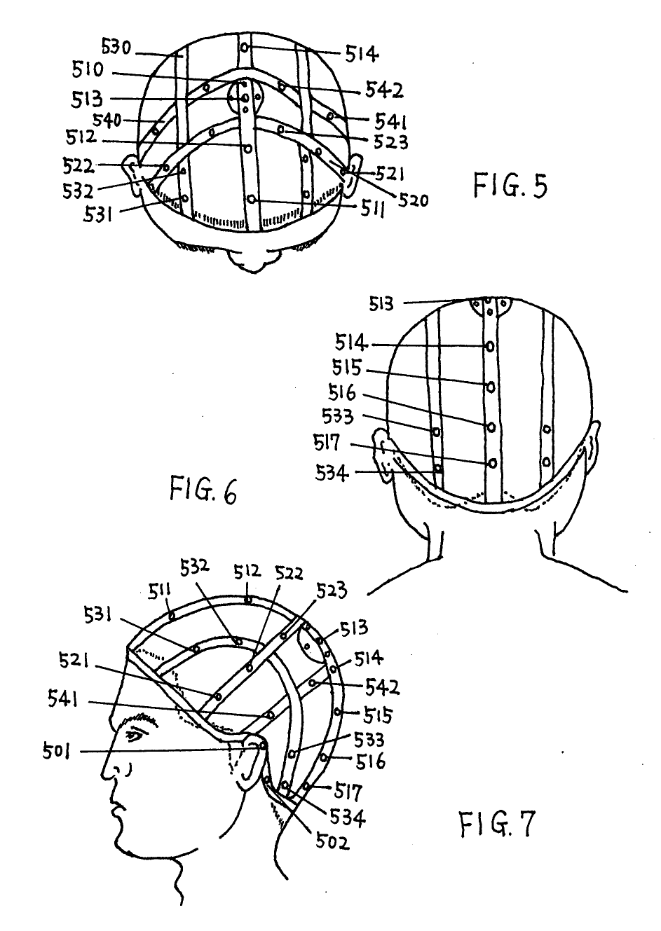

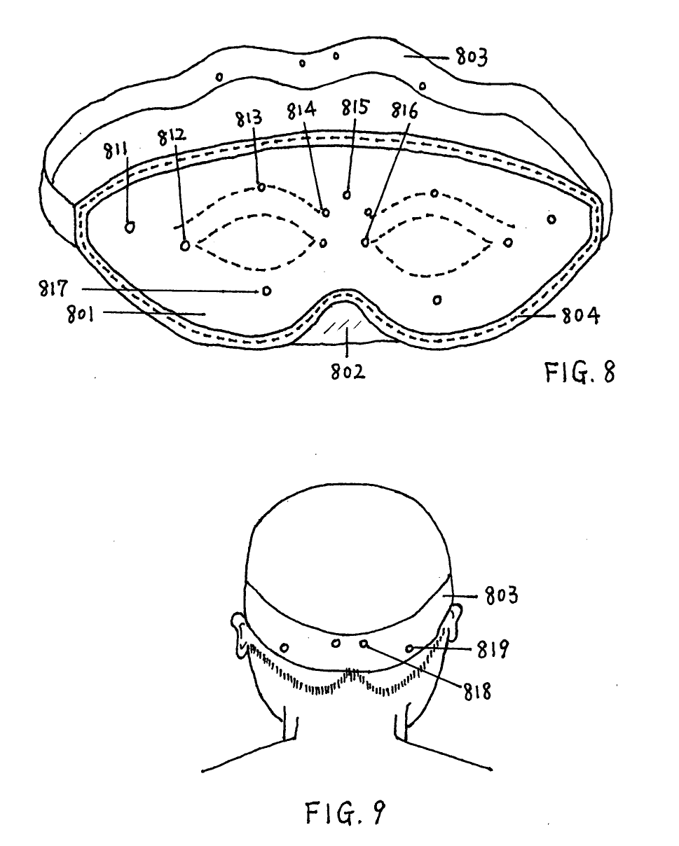

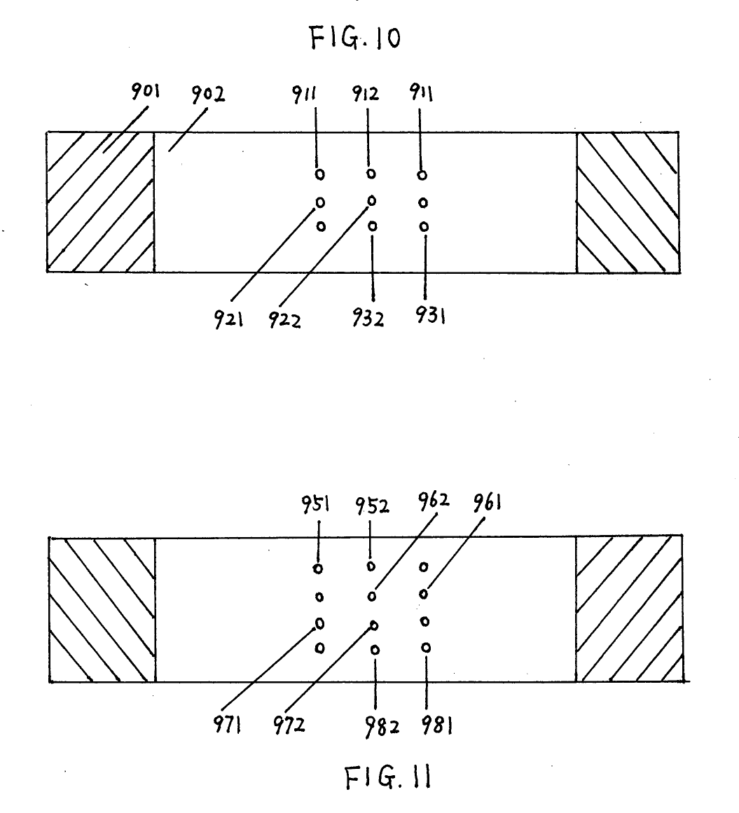

The elastic 102 is a framework that can be made in various types such as the cap (FIG. 5), the eyeshade (FIG. 8), and the waistband (FIG. 10). The elastic 102 make the magnet slices 213 and the electronic pulse contacts 200 to fix easy and touch the human body next to the skin closer together.

FIG. 2 is a illustration of detailed structure about the magnet slices and electronic pulse contacts arrangement. The fixing method of magnet slice adopt “magnetism sandwich method”; 212 is an iron slice having 2 holes. At the point selected as therapy acupoint, fix the iron slice 212 to the elastic 102 with thread and needle, and clip textile 101 with the magnet slice 213 and the iron slice 212 relying on their magnetism.

The fixing method of the electronic pulse contacts adopt “bolt sandwich method”. 200 is a screw-shape electronic pulse contact made in electric conductor; and 202 is its screw; 201 is a screw nut; 203 is a strengthen cushion. The screw 202 pass through the strengthen cushion 203, the nano-micro-biochemical textile 101 and the elastic 102, and fix them with the screw nut 201.

FIG. 3 is a illustration of the electronic pulse device about the medical apparatuses. The electronic pulse device is divided to three parts: (1) the main body 300: comprising the outer covering with the knob of mode 306 and the knob of timer 307; the two control 301 and the two control window 304, 305; the frequency light 302, the low battery indicator 303, and the protection light indicator 308; the display screen of the electronic clock 311 and three button of the electronic clock 312 (it can remind about treating time by self-timer); the two sockets 323; and the interior with one battery (9V size/format) and the collector plate of the electronic pulse device and electronic clock (these techniques existed already). (2) the link wire 320 of the link wire 322 with double plugs 321. (3) another set of accessory 330 having 6-8 electronic pulse contacts 331 that can put on the other acupoints of the human body.

The one link wire with double plugs are very nimble and convenient for the body-thing and the electronic pulse device. Another set of accessory is smarter than before.

The another set of electronic pulse contacts can put on the other therapy acupoints of human body expect the body-thing by the link wire with plugs. It may co-ordinate to treatment of diseases.

FIG. 4 is a illustration of the package bottle and gauze pads soaked with concentrated liquids of herbal medicine. 400 is a bottle and 401 is a bottle stopper. 411 is gauze pads and 421 is concentrated liquid of herbal medicine.

A lot of gauze pads 411 soaked with concentrated liquid of herbal medicine 421. The 411 can put on the surface of the magnet slices 213 and the electronic pulse contacts 200. The gauze pads 411 is very nimble and convenient. The magnet and electronic pulse can enhance absorption function of the herbal medicine.

The herbal medicine liquid can enhance conduction property of electronic pulse contacts. The concentrated liquid of herbal medicine is better than dry powder used by some factory. The absorption action of liquid is better than dry powder.

In first part, the body-thing can work alone by it's function of magnet and nano-micro-biochemical textile. The body-thing can work together with electronic pulse contacts by connecting electronic pulse device and with the gauze pads 411 by putting on the surface of the magnet slices and electronic pulse contacts. The three parts can be divided and work alone or together.

The gauze pads are something that gauze is cut to small round or square slices, and the small slices of the gauze are soaked concentrated liquid of herbal medicine. The concentrated liquid of herbal medicine is made by extracting and concentrating herbs that compound with different herbs based on different formulae. The technique of extraction and concentration is exist already. The different formulae are based on different diseases. Not detail here.

The use methods of the medical apparatuses are very easy. Wear the medical apparatuses such as the cap, the eyeshade and the waistband like general cap, eyeshade and waistband. But they can be connect with electronic pulse device to treat different diseases though electronic pulse on different acupoint. The gauze pads soaked with concentrated liquid of herbal medicine can put on the surface of the magnet slices and the electronic pulse contacts to treat different diseases based on the different formulae of the herbal medicine. The treatment of the electronic pulse and herbal medicine is 1-3 times daily and 15-30 minutes each time. But the cap, the eyeshade and the waistband can be worn more than 1 hour daily based on different medical apparatus. For example, the waistband of losing weight can be worn more than 10 hours daily.

The present invention is now illustrated in greater detail by way of the following examples, but it should be understood that the present invention is not to be construed as being limited thereto.

In the various embodiment of the present invention, the three examples are below.

### EXAMPLE 1

One example of the present invention is a cap of the serials medical apparatuses. FIG. 5 is a illustration of structure and therapy point in front top of the medical cap. FIG. 6 is a illustration of structure and therapy point in back of the medical cap. FIG. 7 is a illustration of structure and therapy point in flank of the medical cap. There are six strip of elastic in the cap. The elastic 500 is around the head, and connects other elastics. The two elastic 520 and 540 are horizontal, from the flank to another flank of the head. The one elastic 510 and two elastics 530 are vertical, from forehead to rear of head.

The elastic 510 is located central line of head, and it belong to Du meridian or governor vessel. 511: ShangXing acupoint (DU23); 512: QianDing acupoint (DU21); 513: BaiHui acupoint (DU20) and SiShenCong acupoints (EX-HN1), (the SiShenCong acupoints are 4 points around the BaiHui acupoint); 514: HouDing acupoint (DU19); 515: QiangJian acupoint (DU18); 516: NaoHu acupoint (DU17); 517: FengFu acupoint (DU16).

The elastic 530 is located the two sides of central line of head. They belong to gallbladder meridian of foot-ShaoYang (GB). 531: TouLinQi acupoint (GB15); 532: MuChuang acupoint (GB16); 533: NaoKong acupoint (GB19); 534: FengChi acupoint (GB20).

The elastic 520 is located the sport field and sense field in scalp acupuncture. 523: the upper part of the sport and sense fields; 152: Middle part of the sport and sense fields; 521: lower part of the sport and sense fields.

The elastic 500 is located the around of head through the forehead and bare of head. 501: JiangYaGo (ditch of reducing blood pressure in ear acupuncture.); 502: AnMian acupoint (point to sleep peacefully).

The electronic pulse contacts are installed at the places of 534, 542, 523 and 521 in the cap of treating apoplexy. The magnet slices are installed at the other places.

### EXAMPLE 2

Another example of the present invention is a eyeshade of the serials medical apparatuses. FIG. 8 is a illustration of structure and therapy point in front of the medical eyeshade. FIG. 9 is a illustration of structure and therapy point in back elastic of the medical eyeshade. 801 is a protagonist of the eyeshade and 803 is a back elastic of the eyeshade. 802 is a nose support and 804 is a sewing edge.

The medical eyeshade is effective for preventing and treating eyestrain syndrome. These acupoint is very important, such as 811: TaiYang acupoint (EX-HN5); 812: TongZiLiao acupoint (GB1); 813: YuYao acupoint (EX-HN4); 814: RanZhu acupoint (BL2); 815: YinTang acupoint (EX-HN3); 816: JingMing acupoint (BL1); 817: ChengQi acupoint (ST1); 818: Vision field (scalp acupuncture); 819: FengChi acupoint (GB20). The electronic pulse contacts are installed at the places of 814, 812, 817 and 818. The magnet slices are installed at the other places.

The main structure and technique of the eyeshade are same as above.

### EXAMPLE 3

Another example of the present invention is a waistband for losing weight in the serials of the medical apparatuses. FIG. 10 is a illustration of structure and therapy point in front part of waistband. FIG. 11 is a illustration of structure and therapy point in back part of the waistband. The waistband is separated to 2 parts or one intact waistband. The separated waistband is more convenient and more precise to fix acupoint and shrink waistline. 901 is velcro on the waistband. 902 is elastic. The acupoints having action of losing weight are called “Eight acupoints around the navel”. 911: HuaRouMen (ST24); 912: ShuiFen (RN9); 931: WaiLing (ST26); 932: YinJiao (RN7); 922: ShenQue (RN8); 921: TianShu (ST25). The therapy acupoints in back part of the waistband are: 951: SanJiaoShu (BL22); 961: ShenShu (BL23); 971: QiHaiShu (BL24); 981: DaChangShu (BL25); 982: YaoYangGuan (DU3); 962: MingMen (DU4); 952: XuanShu (DU5); 972: XiaJiShu (EX-B5). The 912, 921 (2 acupoints), 932 and 951 (2 acupoints), 981 (2 acupoints are fixed with the electronic pulse contacts. The other acupoints are fixed with the magnet slices.

What is claimed is:

1. A system and medical apparatus for preventing and treating diseases by the medical apparatus comprising body-thing, electronic pulse device, medical magnet, herbal medicine gauze pads and nano-micro-biochemical-textile.

2. The system and medical apparatus of claim 1 wherein said the medical apparatus is that comprise three parts in basic structure: (1) body-thing having different size and shape adapted to be worn on the human head or eyes or waist or wrists or arms or legs or knees or shoulder; (2) at least one external electronic pulse device that can be linked with said body-thing; (3) and gauze pads soaked with concentrated liquid of herbal medicine and its use method.

3. The medical apparatus of claim 1 wherein said body-thing is it having different types by the different shape and the different position to be worn on the human body such as head, eyes, waist, wrist, arms, legs, knees or shoulders.

4. The medical apparatus of claim 1 wherein said body-thing is that can be divided three layer: (1) inside layer: the nano-micro-biochemical textile and its accessory—the magnet slices and the electronic pulse contacts put on the therapy acupoints; (2) middle layer: the elastic that make method of fixing the magnet slices and the electronic pulse contacts easy and touch the human body next to the skin closer together; (3) outside layer: the sponge and the adornment cloth.

5. The body-thing of claim 4 wherein said the nano-micro-biochemical-textile is a kind of textile having special biochemical function.

6. The body-thing of claim 4 wherein said the magnet slice is a kind of permanent magnet slice having medical and healthy function. Its diameter is 8-24 millimeter and thickness is 3-8 millimeter. Its magnetic induction intensity is 800-1800 gauss.

7. The body-thing of claim 4 wherein said the method of fixing magnet slice is a kind of magnetism sandwich method. The magnet slice is fixed on the surface of the nano-micro-biochemical-textile by magnetism of another iron slice fixed on the elastic.

8. The body-thing of claim 4 wherein said the electronic pulse contact is a kind of electrode contact made in soft rubber material and shape of screw with head of partial sphere and screw bolt having electric conductivity. Its head diameter is 8-24 millimeter and thickness is 3-8 millimeter. The method of fixing it is by its screw bolt and screw nut with thread. The electronic pulse contact is fixed on the surface of the nano-micro-biochemical-textile by the screw bolt and screw nut and the elastic.

9. The apparatus of claim 1 wherein said the improved electronic pulse device is that is divided to three parts: the main body, the link wire and another set of accessory of electronic pulse contacts.

10. The electronic pulse device of claim 9 wherein said the main body is that comprise the outer covering with the knob of mode and the knob of timer; the two control and the two control window; the frequency light, the low battery indicator, and the protection light indicator; the display screen of the electronic clock and three button of the electronic clock; the two sockets; and the interior with one battery and the collector plate of the electronic pulse device and electronic clock.

11. The electronic pulse device of claim 9 wherein said the link wire is the link wire with double plugs.

12. The electronic pulse device of claim 9 wherein said the another set of accessory of the electronic pulse contact is another 6-8 electronic pulse contacts that can put on the other acupoints of the human body.

13. The medical apparatus of claim 1 wherein said the herbal medicine gauze pads are that gauze is cut to small round or square slice pads soaked with concentrated liquid of herbal medicine.

14. The medical apparatus of claim 2 wherein said the concentrated liquid of herbal medicine is the liquid that is made by extracting and concentrating herbal medicines, and said the use method is that the gauze pads can be put on surface of the magnet slices and electronic pulse contacts in the body-thing.

15. The medical apparatus of claim 1 wherein said the herbal medicine is that compound with different herbs based on different formulae. The different formulae are based on the need of treating different diseases.

16. The magnet slices and electronic pulse contacts of claim 4 wherein said the therapy acupoints are ShangXing, QianDing, BaiHui, SiShenCong, HouDing, QiangJian, NaoHu, FengFu, TouLinQi, MuChuang, NaoKong, FengCi in the medical cap.

17. The magnet slices and electronic pulse contacts of claim 4 wherein said the therapy acupoints are TaiYang, TongZiLiao, YuYao, RanZhu, YinTang, JingMing, ChengQi, Vision field, and FengChi in the medical eyeshade.

18. The magnet slices and electronic pulse contacts of claim 4 wherein said the therapy acupoints are HuaRouMen, ShuiFen, WaiLing, YinJiao, ShenQue, TianShu, SanJiaoShu, ShenShu, QiHaiShu, DaChangShu, YaoYangGuan, MingMen, XuanShu, XiaJiShu in the medical waistband.
